(19) United States
(12) Patent Application Publication
(10) Pub. No.: US 2009/0143491 A1
(43) Pub. Date: Jun. 4, 2009

(54) PROCESS FOR STABILISING A CATALYST

(76) Inventors: Peter John VAN DEN BRINK, Amsterdam (NL); Hendrik Albertus Colijn, Amsterdam (NL); Thomas Joris Remans, Amsterdam (NL)

Correspondence Address: SHELL OIL COMPANY, P O BOX 2463, HOUSTON, TX, 772522463, US

(21) Appl. No.: 12/208,172

(22) Filed: Sep. 10, 2008

(30) Foreign Application Priority Data
Sep. 10, 2007 (EP) 07115989.1

(51) Int. Cl. C07C 27/10; B01J 31/12

(52) U.S. Cl. 518/700; 502/158

(57) ABSTRACT

The invention provides a process for modifying a Fischer-Tropsch catalyst or catalyst precursor, the process comprising the steps of: contacting a Fischer-Tropsch catalyst or catalyst precursor comprising a titania carrier with a compound having the general formula RₐX₍₄₋ₐ₎Y wherein a is in the range of 1-3, and wherein the or each R is independently an alkyl or aryl group; Y is silicon or titanium, and, the or each X is independently chosen from the group consisting of hydrogen, an alkoxy group and ZY′BB′B^{2′}, Z being oxygen, —NH, or —NR, wherein R is an alkyl or aryl group comprising 1 to 8 carbon atoms, Y′ being silicon or titanium, and B, B′ and B² ,independently being hydrogen or an alkyl, aryl, or alkoxy group.

In a preferred embodiment the compound RₐX₍₄₋ₐ₎Y is hexamethyldisilazane.

This application claims the benefit of European Application No. 07115989.1 filed Sep. 10, 2007.

### BACKGROUND OF THE INVENTION

This invention relates to a process for improving the hydrothermal stability of a Fischer-Tropsch catalyst or catalyst precursor.

Many documents are known describing processes for the catalytic conversion of (gaseous) hydrocarbonaceous feedstocks, especially methane, natural gas and/or associated gas, into liquid products, especially methanol and liquid hydrocarbons, particularly paraffinic hydrocarbons.

The Fischer-Tropsch process can be used as part of the conversion of hydrocarbonaceous feedstocks into liquid and/or solid hydrocarbons. Generally the feedstock (e.g. natural gas, associated gas and/or coal-bed methane, coal) is converted in a first step into a mixture of hydrogen and carbon monoxide (this mixture is often referred to as synthesis gas or syngas). The synthesis gas is then fed into a reactor where it is converted in one or more steps over a suitable catalyst at elevated temperature and pressure into compounds ranging from methane to high molecular weight modules comprising up to 200 carbon atoms, or, under particular circumstances, even more.

Catalysts used in the Fischer-Tropsch synthesis often comprise a carrier material and one or more metals selected from Groups 8-10 of the Periodic Table of Elements, especially from the cobalt and iron groups, optionally in combination with one or more metal oxides and/or metals as promoters selected from zirconium, titanium, chromium, vanadium and manganese, especially manganese. Such catalysts are known in the art and have been described for example, in the specifications of WO 9700231A and U.S. Pat. No. 4,595,703.

The carrier based support material may be a refractory oxide. One particularly suitable carrier based support material for Fischer-Tropsch catalysts is titania. As an example of a catalyst suitable for Fischer-Tropsch reactions can be mentioned a catalyst comprising cobalt in titania. Typically at least 50% of the titania is in the anatase crystal form, which exhibits the largest surface area. The catalyst/catalyst precursor is normally, but not always, calcined.

A by-product of the Fischer-Tropsch reaction is water which results in water vapour contacting the catalyst which consequently suffers from sintering and agglomeration of support particles thus reducing the surface area. Water also causes anatase titania crystals to convert into the rutile crystalline form (which have a smaller surface area) and oxidises the active metal to a metal hydroxide.

### SUMMARY OF THE INVENTION

According to one aspect of the present invention, there is provided a process for modifying a Fischer-Tropsch catalyst or catalyst precursor, the process comprising the steps of:
(a) contacting a Fischer-Tropsch catalyst or catalyst precursor comprising a titania carrier, a catalytically active metal or precursor therefor, and optionally a promoter or precursor therefor, with a compound having the general formula: RₐX₍₄₋ₐ₎Y
   wherein a is in the range of 1-3, preferably a=3, and
   wherein the or each R is independently an alkyl or aryl group comprising 1 to 8 carbon atoms,
   Y is silicon or titanium, and
   the or each X is independently chosen from the group consisting of hydrogen, an alkoxy group comprising 1 to 8 carbon atoms, and ZY′BB′B^{2′};
   Z being oxygen, —NH, or —NR, wherein R is an alkyl or aryl group comprising 1 to 8 carbon atoms,
   Y′ being silicon or titanium, and
   B, B′ and B^{2′}, independently being hydrogen or an alkyl, aryl, or alkoxy group comprising 1 to 8 carbon atoms;
(b) keeping the temperature of the resulting modified catalyst or catalyst precursor below 400° C., preferably below 350° C., more preferably below 300° C., up to the moment the resulting catalyst or catalyst precursor is subjected to an activation step or used in a Fischer-Tropsch reaction.

### DETAILED DESCRIPTION OF THE INVENTION

For the present invention, a Fischer-Tropsch catalyst precursor is defined as a catalyst which after treatment with hydrogen or a hydrogen comprising gas, i.e. after activation, can be used as catalyst in a Fischer-Tropsch reaction.

The Fischer-Tropsch catalyst or catalyst precursor comprises a titania carrier. Preferably more than 70 weight percent of the carrier material consists of titania, more preferably more than 80 weight percent, most preferably more than 90 weight percent, calculated on the total weight of the carrier material. The remainder may, for example, be a different type of refractory oxide. Typically at least 50% of the titania is in the anatase crystal form, which exhibits the largest surface area. The catalyst or catalyst precursor is normally, but not always, calcined. If calcined, it is calcined before the modifying process of the present invention.

In the process according to the present invention a Fischer-Tropsch catalyst or catalyst precursor comprising a titania carrier is contacted with a compound having the general formula RₐX₍₄₋ₐ₎Y, with R, X, a and Y as specified above. Without wishing to be bound by any theory, the process of the present invention seems to allow the RₐX₍₄₋ₐ₎Y compound to bond to the surface of the titania. It seems that the RₐX₍₄₋ₐ₎Y compound bonds to oxygen on the surface of the titania to form —O—Y—RₐX₍₃₋ₐ₎. It seems that the RₐX₍₄₋ₐ₎Y compound reacts with —OH groups on the surface.

When a=3, for example, the RₐX₍₄₋ₐ₎Y compound reacts with —OH groups on the surface to form —O—Y—R₃. For example, when (CH₃)₃Si—NH—Si(CH₃)₃ reacts with the titania surface, —O—Si(CH₃)₃ groups are formed on the surface. Similarly, when Si(CH₃)₃OCH₃ reacts with the titania surface, —O—Si(CH₃)₃ groups are formed on the surface.

It has been found that with the process of the current invention, using a compound having the general formula RₐX₍₄₋ₐ₎Y, with R, a, Z, and Y as defined above, it is relatively easy to form —O—Y—RₐX₍₃₋ₐ₎ groups on the titania surface. On the other hand, once formed, these —O—Y—RₐX₍₃₋ₐ₎ groups show a good stability under reduction conditions and under Fischer-Tropsch conditions.

The presence of such groups seems to reduce the hydrophilic properties of the surface of the titania. It has been found that after the process of the present invention, the problems encountered in situ in the reactor with water are reduced. The amount of sintering and agglomeration of the titania particles and other associated problems is reduced. Less anatase is converted to rutile, and less oxidation of cobalt to cobalt oxide and cobalt hydroxide takes place. This is in marked contrast to EP 0180269, which teaches how to resist reaction of the active metal of a catalyst with the carrier material.

The process of the current invention shows better results than a process in which a compound comprising halogen, such as trimethylsilyl chloride, is used. It was found that halogen-comprising compounds react too slowly with the titania surface. Furthermore, halogen compounds result in the corrosion of the steel inside a Fischer-Tropsch reactor.

It has been found that compounds like Si(OCH₃)₄ react too fast with the titania surface, and —O—Si(OCH₃)₃ groups do not show a good stability under reduction conditions and under Fischer-Tropsch conditions.

In a process according to the present invention, X may be alkoxy. In that case, the compound RₐX₍₄₋ₐ₎Y comprises at most three alkoxy groups per silicon or titanium atom. After reaction with the titania surface, the group attached to the oxygen on the titania surface comprises at most two alkoxy groups. In a preferred embodiment, the total number of alkyl plus aryl groups in the compound RₐX₍₄₋ₐ₎Y is equal or larger than the number of alkoxy groups per silicon or titanium atom. In other words, in case X is alkoxy, a preferably is 2 or 3.

Similarly, in case X is ZY′BB′B^{2′}, B, B′ and B^{2′} may all three represent an alkoxy group. Preferably at least one is an alkyl or aryl group. More preferably B, B′ and B^{2′} independently are an alkyl or aryl group comprising 1 to 8 carbon atoms.

After contacting a Fischer-Tropsch catalyst or catalyst precursor comprising a titania carrier, with a compound RₐX₍₄₋ₐ₎Y, the temperature of the resulting modified catalyst or catalyst precursor should be kept below 400° C. The temperature of the modified catalyst or catalyst precursor should not be brought above 400° C. in order to avoid decomposition of the —O—Y—RₐX₍₃₋ₐ₎ groups on the surface. For example, if —O—Si(CH₃)₃ groups have been formed on the surface, they should not decompose to SiO₂.

Preferably a modified catalyst or catalyst precursor is kept below 350° C., more preferably below 300° C., even more preferably below 180° C., most preferably below 150° C., up to the moment the resulting catalyst or catalyst precursor is subjected to an activation step or used in a Fischer-Tropsch reaction.

Activation and/or Fischer-Tropsch synthesis normally are performed at a temperature below 400° C. Typically activation, i.e. reduction, takes place at temperatures of about 200° to 350° C. The Fischer-Tropsch synthesis is preferably carried out at a temperature in the range from 125 to 350° C., more preferably 175 to 275° C., most preferably 200 to 260° C.

The catalyst or catalyst precursor may be shaped prior to modification according to the present invention. It may, for example, be formed from a catalyst or catalyst precursor material. Methods of preparing a shaped catalyst carrier include spray drying, (wheel) pressing, extruding or otherwise forcing a granular or powdered catalyst or catalyst precursor material into various shapes under certain conditions, which will ensure that the particle retains the resulting shape.

Alternatively, the catalyst or catalyst precursor may be prepared using coating processes, e.g. spray coating, dip-coating or painting.

A preferred method for preparing a catalyst or catalyst precursor that can be modified according to our invention is by extrusion, especially if the catalyst is to be applied in a fixed bed reactor. If the catalyst is to be used in a slurry reactor the catalyst or catalyst precursor is preferably prepared by spray drying.

When a shaped catalyst or catalyst precursor is prepared, it may be advantageous to add a binder material, for example to increase the mechanical strength of the catalyst or catalyst precursor. The shaped catalyst or catalyst precursor may suitably comprise up to 30 wt % of another refractory oxide, typically amorphous silica, alumina or zirconia, organic glues, a clay or combinations thereof as a binder material, preferably up to 20% by weight based on the total weight of titania and binder material. More preferably a silica and alumina mixture is used as binder where the binder makes up less than about 30 wt %, preferably less than about 20 wt %, more preferably about 3-20 wt %, still more preferably 4-15 wt %, yet more preferred 5-10 wt % based on the total weight of titania and binder material.

In order to achieve the benefits of porosity and strength, binder materials may be mixed with the titania starting material before the shaping operation. Binder materials may be added in a variety of forms, as salts or preferably as colloidal suspensions or sols. For example, alumina sols made from aluminium chloride, acetate, or nitrate are preferred sources of the alumina component. Readily available silica sols are preferred sources of the silica component.

When a shaped catalyst or catalyst precursor is prepared, the process may comprise the following steps: (a) mixing (1) titania, (2) a liquid, and (3) a compound containing a metal selected from Groups 8-10 of the Periodic Table of Elements, which is at least partially insoluble in the amount of liquid used, to form a mixture,(b) shaping the mixture thus-obtained, (c) drying, and (d) optionally calcining.

References to “Groups” and the Periodic Table as used herein relate to the new IUPAC version of the Periodic Table of Elements such as that described in the 87^{th} Edition of the Handbook of Chemistry and Physics (CRC Press).

The liquid may be any of suitable liquids known in the art, for example water; ammonia; alcohols, such as methanol, ethanol and propanol; ketones, such as acetone; aldehydes, such as propanal and aromatic solvents, such as toluene. A most convenient and preferred liquid is water.

To improve the flow properties of the mixture, it is preferred to include one or more flow improving agents and/or extrusion aids in the mixture prior to extrusion. Suitable additives for inclusion in the mixture include fatty amines, quaternary ammonium compounds, polyvinyl pyridine, sulphoxonium, sulphonium, phosphonium and iodonium compounds, alkylated aromatic compounds, acyclic mono-carboxylic acids, fatty acids, sulphonated aromatic compounds, alcohol sulphates, ether alcohol sulphates, sulphated fats and oils, phosphonic acid salts, polyoxyethylene alkylphenols, polyoxyethylene alcohols, polyoxyethylene alkylamines, polyoxyethylene alkylamides, polyacrylamides, polyols and acetylenic glycols. Preferred additives are sold under the trademarks Nalco and Superfloc.

To obtain strong extrudates, it is preferred to include in the mixture, prior to extrusion, at least one compound which acts as a peptising agent for the titania. Suitable peptising agents for inclusion in the extrudable mixture are well known in the art and include basic and acidic compounds. Examples of basic compounds are ammonia, ammonia-releasing compounds, ammonium compounds or organic amines. Such basic compounds are removed upon calcination and are not retained in the extrudates to impair the catalytic performance of the final product. Preferred basic compounds are organic amines or ammonium compounds. A most suitable organic amine is ethanol amine. Suitable acidic peptising agents include weak acids, for example formic acid, acetic acid, citric acid, oxalic acid, and propionic acid.

Optionally, burn-out materials may be included in the mixture, prior to extrusion, in order to create macropores in the resulting extrudates. Suitable burn-out materials are commonly known in the art.

The total amount of flow-improving agents/extrusion aids, peptising agents, and burn-out materials in the mixture preferably is in the range of from 0.1 to 20% by weight, more preferably from 0.5 to 10% by weight, on the basis of the total weight of the mixture. Examples of suitable catalyst preparation methods as described above are disclosed in WO-A-9934917.

Preferably the process is performed on a catalyst precursor, particularly one comprising titania and active metal precursor in its oxidised form. For example, the process may be performed on a catalyst precursor comprising titania and cobalt oxide, and optionally a promoter. Optionally the catalyst precursor has been calcined although for certain embodiments calcination is not required at any point. A catalyst precursor, optionally calcined, that is treated according to the present inventor can be placed in the reactor, and subsequently reduced.

The Fischer-Tropsch catalyst or catalyst precursor is contacted with a compound having the general formula RₐX₍₄₋ₐ₎Y. The, or each, R is independently an alkyl or aryl group comprising 1 to 8 carbon atoms; and a is in the range of 1-3. Y is silicon or titanium. The, or each, X is independently chosen from the group consisting of hydrogen, an alkoxy group comprising 1 to 8 carbon atoms and ZY′BB′B^{2′}, Z being oxygen, —NH, or —NR, wherein R is an alkyl or aryl group comprising 1 to 8 carbon atoms, Y′ being silicon or titanium, and B, B′ and B^{2′}, independently being hydrogen or an alkyl, aryl, or alkoxy group comprising 1 to 8 carbon atoms.

Preferably Y is Si. Y and Y′ may be the same or different. If Y′ is present, preferably Y and Y′ are the same. If Y′ is present, most preferably Y and Y′ are Si.

Preferably the or each R group in RₐX₍₄₋ₐ₎Y is an alkyl group comprising 1 to 8 carbon atoms, more preferably an alkyl group comprising 1 to 3 carbon atoms, such as a —CH₃ group or a —CH2CH3 group. Preferably a=3.

For one embodiment a=3 and each R is a —CH₃ group.

For certain embodiments, X is hydrogen or an alkoxy group, especially an alkoxy group. In one embodiment the compound comprises Si(CH₃)₃X wherein X is hydrogen or an alkoxy group.

For certain preferred embodiments, the compound RₐX₍₄₋ₐ₎Y is Si(CH₃)₃OR, wherein R is alkyl group comprising 1 to 8 carbon atoms, preferably 1 to 3 carbon atoms. In a preferred embodiment the compound RₐX₍₄₋ₐ₎Y is Si(CH₃)₃OCH₃.

For certain preferred embodiments, the compound RₐX₍₄₋ₐ₎Y is Si(CH₂CH₃)₃OR, wherein R is alkyl group comprising 1 to 8 carbon atoms, preferably 1 to 3 carbon atoms. In a preferred embodiment the compound RₐX₍₄₋ₐ₎Y is Si(CH₂CH₃)₃OCH₃.

Preferably the total number of carbon atoms in the compound is at least 3. Where the total number of carbon atoms in Ra is less than three, preferably X comprises B, B′, and B^{2′} at least one of which comprises carbon atoms sufficient to increase the total number of carbon groups in the compound to more than 3, preferably more than 5.

For certain embodiments X may be a ZY′-BB′B^{2′} group. The process thus typically allows the Y′-BB′B^{2′} groups to bond to the surface of the titania as described above for the Y—RₐX₍₃₋ₐ₎ groups. Hence, when X is ZY′-BB′B^{2′}, both —O—Y—RₐX₍₃₋ₐ₎ groups and —O—Y′-BB′B^{2′} groups may be formed on the titania surface. In case a=3, —O—Y—R₃ groups and —O—Y′-BB′B^{2′} groups may be formed on the titania surface.

Preferably a=3 and X is ZY′-BB′B^{2′}.

Preferably Z is —NH or —NR, wherein R is an alkyl or aryl group comprising 1 to 8 carbon atoms. If Z is —NR, preferably R is an alkyl group comprising 1 to 3 carbon atoms, more preferably methyl. Most preferably Z is —NH.

Preferably B, B′, and B^{2′} are each independently an alkyl, aryl, alkoxy group comprising 1 to 8 carbon atoms. More preferably B, B′, and B^{2′} are each independently an alkyl or aryl group comprising 1 to 8 carbon atoms, even more preferably an alkyl group comprising 1 to 3 carbon atoms; especially an alkyl group such as an ethyl or a methyl group, particularly a methyl group.

Preferably the compound RₐX₍₄₋ₐ₎Y is R₃Y′—ZBB′B^{2′}Y, more preferably R₃Si—NH—SiBB′B^{2′}. In a preferred embodiment the compound RₐX₍₄₋ₐ₎Y is R₃Si—NH—SiR₃, wherein R is an alkyl or aryl group comprising 1 to 8 carbon atoms; preferably R is an alkyl group comprising 1 to 3 carbon atoms, more preferably R is an ethyl or methyl group, particularly a methyl group.

A preferred embodiment comprises hexamethyldisilazane, i.e. (CH₃)₃Si—NH—Si(CH₃)₃. An alternative embodiment comprises di(trimethlysilyl) oxide.

The Fischer-Tropsch catalyst or catalyst precursor can be contacted with the compound having the general formula RₐX₍₄₋ₐ₎Y either in the gaseous phase or in the liquid phase. The preferred contact time is 1-90 minutes, preferably 15-45 minutes. The compound may be provided as a liquid—such as in a slurry, or a solution, or the compound may be a liquid under ambient or raised temperatures. Alternatively, the compound may be in the gas phase when contacted with the catalyst or catalyst precursor.

The process of the invention is preferably carried out using a solution of the compound in an organic solvent preferably containing no hydroxyl groups. Suitable solvents are octane, benzene, toluene, xylene, aceonitrile and dimethyl sulfoxide; especially toluene. Preference is given to the use of a hydrocarbon or a mixture of hydrocarbons as the solvent.

When the compound having the general formula RₐX₍₄₋ₐ₎Y is provided as a liquid, the process of the invention is preferably carried out at a temperature of 40-200° C. and in particular of 80-149° C., such as around 144° C. When the process is carried out using a solution of the compound in a solvent, the temperature used preferably is within 10° C. of the boiling point of the solvent.

When the compound having the general formula RₐX₍₄₋ₐ₎Y is in the gas phase, the temperatures can be higher although preferably not high enough to cause the compound to decompose. Preferably the temperature does not exceed 350° C.

Preferably the catalyst or catalyst precursor is not calcined following contact with the compound RₐX₍₄₋ₐ₎Y. Preferably the catalyst or catalyst precursor is not heated to above 400° C., more preferably the modified catalyst or catalyst precursor is not heated to above 350° C. Nevertheless typically the resulting modified catalyst or catalyst precursor is heated to relatively milder temperatures (for example up to 149° C.) in order to remove the solvent from the catalyst or catalyst precursor. Preferably the solvent is removed before the modified catalyst or catalyst precursor is reduced and/or put into use and/or treated to add, for example, a promoter or a catalytically active metal.

In a preferred embodiment, the catalyst or catalyst precursor is contacted with a mixture comprising H₂O in the form of water or water vapour, preferably water vapour, before contact with the compound. As mentioned above, the catalyst or catalyst precursor may be prepared by (a) mixing titania, a liquid, and a compound containing a metal selected from Groups 8-10 of the Periodic Table of Elements, (b) shaping the mixture thus-obtained, (c) drying, and (d) optionally calcining.

In that case the catalyst is preferably contacted with a mixture comprising H₂O in the form of water or water vapour following drying and/or calcination.

Contacting the catalyst or catalyst precursor with a mixture comprising H₂O in the form of water or water vapour seems to increase the proportion of surface —OH groups which in turn increase the number of Y—RₐX₍₃₋ₐ₎ groups, and where present, Y-BB′B^{2′} groups, which can bond to the surface of the titania during the subsequent reaction with the compound.

Typically pure water is used although, for example, a small amount of alcohol may be added.

Preferably said mixture comprising water is contacted with the catalyst or catalyst precursor whilst at a temperature of between 50-200° C., preferably 50-149° C., more preferably 70-110° C., especially around 90° C.

Following such water treatment the catalyst or catalyst precursor preferably is dried at a temperature of 120-160° C., more preferably at a temperature of 120-149° C., even more preferably around 140° C.

A Fischer-Tropsch catalyst or catalyst precursor that can be treated in accordance with the present invention comprises a titania carrier, a catalytically active metal or precursor therefor, and optionally a promoter or precursor therefor. Typically the active metal is one or more selected from the group consisting of: cobalt, iron, nickel and ruthenium; preferably cobalt.

In one way to prepare a cobalt comprising catalyst or catalyst precursor, cobalt hydroxide (Co(OH)₂) can be used as a starting material. This material is usually mixed with one or more co-catalysts, promoters, etc, and a carrier (in this case titania or a mixture comprising titania), and then calcined. During calcination cobalt oxide (CoO) is formed, and next the cobalt is further oxidised to form Co₃O₄. The calcined catalyst or catalyst precursor normally is placed in a Fischer-Tropsch reactor. In the reactor the cobalt oxide is reduced to cobalt.

Typically, the amount of catalytically active metal in the catalyst or catalyst precursor may range from 1 to 100 parts by weight per 100 parts by weight of carrier material, preferably from 3 to 50 parts by weight per 100 parts by weight of carrier material.

Typically the promoter(s) and/or co-catalyst(s) are one or more selected from the group consisting of: titanium, zirconium, manganese, vanadium, rhenium, platinum and palladium, or an oxide thereof, or a combination of one or more of said metals or their oxides; preferably manganese or vanadium.

Suitable metal oxide promoters may be selected from Groups 2-7 of the Periodic Table of Elements, or the actinides and lanthanides. In particular, oxides of magnesium, calcium, strontium, barium, scandium, yttrium, lanthanum, cerium, titanium, zirconium, hafnium, thorium, uranium, vanadium, chromium and manganese are most suitable promoters.

Suitable metal promoters may be selected from Groups 7-10 of the Periodic Table. Manganese, iron, rhenium and Group 8-10 noble metals are particularly suitable, with platinum and palladium being especially preferred. The amount of promoter present in the catalyst is suitably in the range of from 0.01 to 100 pbw, preferably 0.1 to 40, more preferably 1 to 20 pbw, per 100 pbw of carrier material.

The catalyst may comprise a promoter(s) and/or co-catalyst(s) having a concentration in the catalyst material in the range 1-20 atom % of the active metal, preferably 3-7 atom %, and more preferably 4-6 atom %.

The catalytically active material could also be present with one or more co-catalysts. Suitable co-catalysts include one or more metals such as iron, nickel, or one or more noble metals from Groups 8-10. Preferred noble metals are platinum, palladium, rhodium, ruthenium, iridium and osmium.

A suitable catalyst comprises cobalt as the catalytically active metal and zirconium as a promoter and titania as the carrier material.

Another suitable catalyst comprises cobalt as the catalytically active metal and manganese and/or vanadium as a promoter and titania as the carrier material.

Activation of a fresh prepared catalyst precursor can be carried out in any known manner and under conventional conditions. For example, the catalyst precursor may be activated by contacting it with hydrogen or a hydrogen-containing gas, typically at temperatures of about 200° to 350° C.

The present invention also provides a Fischer-Tropsch catalyst treated according to the process as described herein.

The present invention also provides a process for the production of liquid hydrocarbons from synthesis gas, the process comprising:
converting synthesis gas in a reactor into liquid hydrocarbons, and optionally solid hydrocarbons and optionally liquefied petroleum gas, at elevated temperatures and pressures; using a catalyst treated as described herein.

The Fischer-Tropsch process is well known to those skilled in the art and involves synthesis of hydrocarbons from syngas, by contacting the syngas at reaction conditions with the Fischer-Tropsch catalyst.

The synthesis gas can be provided by any suitable means, process or arrangement. This includes partial oxidation and/or reforming of a hydrocarbonaceous feedstock as is known in the art.

Typically the synthesis gas is produced by partial oxidation of a hydrocarbonaceous feed. The hydrocarbonaceous feed suitably is methane, natural gas, associated gas or a mixture of C₁₋₄ hydrocarbons. The feed comprises mainly, i.e. more than 90 v/v %, especially more than 94%, C₁₋₄ hydrocarbons, especially comprises at least 60 v/v percent methane, preferably at least 75 percent, more preferably 90 percent. Very suitably natural gas or associated gas is used. As described above, any sulphur in the feedstock is preferably removed or at least minimised.

The partial oxidation of gaseous feedstocks, producing mixtures of especially carbon monoxide and hydrogen, can take place according to various established processes. These processes include the Shell Gasification Process. A comprehensive survey of this process can be found in the Oil and Gas Journal, Sep. 6, 1971, pp 86-90.

The oxygen containing gas for the partial oxidation typically contains at least 95 vol. %, usually at least 98 vol. %, oxygen. Oxygen or oxygen enriched air may be produced via cryogenic techniques, but could also be produced by a membrane based process, e.g. the process as described in WO 93/06041. A gas turbine can provide the power for driving at least one air compressor or separator of the air compression/separating unit. If necessary, an additional compressing unit may be used after the separation process, and the gas turbine in that case may also provide at the (re)start power for this compressor. The compressor, however, may also be started at a later point in time, e.g. after a full start, using steam generated by the catalytic conversion of the synthesis gas into hydrocarbons.

To adjust the H₂/CO ratio in the syngas, carbon dioxide and/or steam may be introduced into the partial oxidation process. Preferably up to 15% volume based on the amount of syngas, preferably up to 8% volume, more preferable up to 4% volume, of either carbon dioxide or steam is added to the feed. Water produced in the hydrocarbon synthesis may be used to generate the steam. As a suitable carbon dioxide source, carbon dioxide from the effluent gasses of the expanding/combustion step may be used. The H₂/CO ratio of the syngas is suitably between 1.5 and 2.3, preferably between 1.6 and 2.0. If desired, (small) additional amounts of hydrogen may be made by steam methane reforming, preferably in combination with the water gas shift reaction.

The syngas comprising predominantly hydrogen, carbon monoxide and optionally nitrogen, carbon dioxide and/or steam is contacted with a suitable catalyst in the catalytic conversion stage, in which the hydrocarbons are formed. Suitably at least 70 v/v % of the syngas is contacted with the catalyst, preferably at least 80%, more preferably at least 90%, still more preferably all the syngas.

The Fischer-Tropsch synthesis is preferably carried out at a temperature in the range from 125 to 350° C., more preferably 175 to 275° C., most preferably 200 to 260° C. The pressure preferably ranges from 5 to 150 bar abs., more preferably from 5 to 80 bar abs.

The Fischer-Tropsch process can be carried out in a slurry phase regime or an ebullating bed regime, wherein the catalyst particles are kept in suspension by an upward superficial gas and/or liquid velocity.

Another regime for carrying out the Fischer-Tropsch process is a fixed bed regime, especially a trickle flow regime. A very suitable reactor is a multitubular fixed bed reactor. In addition, the Fischer-Tropsch process may also be carried out in a fluidised bed process.

The catalyst according to the present invention may be used in any type of Fischer-Tropsch reactor.

Products of the Fischer-Tropsch synthesis may range from methane to heavy paraffin waxes. Preferably, the production of methane is minimised and a substantial portion of the hydrocarbons produced have a carbon chain length of a least 5 carbon atoms. Preferably, the amount of C₅₊ hydrocarbons is at least 60% by weight of the total product, more preferably, at least 70% by weight, even more preferably, at least 80% by weight, most preferably at least 85% by weight.

The hydrocarbons produced in the process are suitably C₃₋₂₀₀ hydrocarbons, more suitably C₄₋₁₅₀ hydrocarbons, especially C₅₋₁₀₀ hydrocarbons, or mixtures thereof. These hydrocarbons or mixtures thereof are liquid or solid at temperatures between 5 and 30° C. (1 bar), especially at about 20° C. (1 bar), and usually are paraffinic of nature, while up to 30 wt %, preferably up to 15 wt %, of either olefins or oxygenated compounds may be present.

Depending on the catalyst and the process conditions used in a Fischer-Tropsch reaction, various proportions of normally gaseous hydrocarbons, normally liquid hydrocarbons and optionally normally solid hydrocarbons are obtained. It is often preferred to obtain a large fraction of normally solid hydrocarbons. These solid hydrocarbons may be obtained up to 90 wt % based on total hydrocarbons, usually between 50 and 80 wt %.

A part may boil above the boiling point range of the so-called middle distillates. The term “middle distillates”, as used herein, is a reference to hydrocarbon mixtures of which the boiling point range corresponds substantially to that of kerosene and gasoil fractions obtained in a conventional atmospheric distillation of crude mineral oil. The boiling point range of middle distillates generally lies within the range of about 150 to about 360° C.

The higher boiling range paraffinic hydrocarbons, if present, may be isolated and subjected to a catalytic hydrocracking step, which is known per se in the art, to yield the desired middle distillates. The catalytic hydro-cracking is carried out by contacting the paraffinic hydrocarbons at elevated temperature and pressure and in the presence of hydrogen with a catalyst containing one or more metals having hydrogenation activity, and supported on a support comprising an acidic function. Suitable hydrocracking catalysts include catalysts comprising metals selected from Groups 6 and 8-10 of the (same) Periodic Table of Elements. Preferably, the hydrocracking catalysts contain one or more noble metals from Groups 8-10. Preferred noble metals are platinum, palladium, rhodium, ruthenium, iridium and osmium. Most preferred catalysts for use in the hydro-cracking stage are those comprising platinum.

The amount of catalytically active noble metal present in the hydrocracking catalyst may vary within wide limits and is typically in the range of from about 0.05 to about 5 parts by weight per 100 parts by weight of the support material. The amount of non-noble metal present is preferably 5-60%, preferably 10-50%.

Suitable conditions for the catalytic hydrocracking are known in the art. Typically, the hydrocracking is effected at a temperature in the range of from about 175 to 400° C. Typical hydrogen partial pressures applied in the hydrocracking process are in the range of from 10 to 250 bar.

The product of the hydrocarbon synthesis and consequent hydrocracking suitably comprises mainly normally liquid hydrocarbons, beside water and normally gaseous hydrocarbons. By selecting the catalyst and the process conditions in such a way that especially normally liquid hydrocarbons are obtained, the product obtained (“syncrude”) may be transported in the liquid form or be mixed with any stream of crude oil without creating any problems as to solidification and or crystallization of the mixture. It is observed in this respect that the production of heavy hydrocarbons, comprising large amounts of solid wax, are less suitable for mixing with crude oil while transport in the liquid form has to be done at elevated temperatures, which is less desired.

Thus according to a further aspect of the invention, there is provided hydrocarbon products synthesised by a Fischer-Tropsch reaction and catalysed by a catalyst treated as described and herein.

The hydrocarbon products may have undergone the steps of hydroprocessing, preferably hydrogenation, hydroisomerisation and/or hydrocracking. In particular, the hydrocarbon products may comprise a fuel, preferably naphtha, kerosene or gasoil, a waxy raffinate or a base oil.

Any percentage mentioned in this description is calculated on total weight or volume of the composition, unless indicated differently. When not mentioned, percentages are considered to be weight percentages. Pressures are indicated in bar absolute, unless indicated differently.

Embodiments of the present invention will now be described, by way of example only.

### EXAMPLES

### Preparation of Catalyst or Catalyst Precursor Samples

Various Fischer-Tropsch catalyst or catalyst precursor samples were prepared by mixing Co/Mn hydroxide co-precipitate with titania (P25 available from Degussa™), citric acid, flocculent and water until a paste was obtained. The paste was kneaded and compacted before being extruded and then calcined at 580° C.

The resulting samples comprised a titania carrier, manganese promoter and cobalt oxide, being the precursor to the active metal.

### Test Method

As the skilled person will appreciate, the main Fischer-Tropsch reaction (CO+H₂−>hydrocarbons) produces water. Thus to simulate such conditions the various samples were treated with water in an autoclave at similar conditions to that found in a Fischer-Tropsch reactor. Indeed the autoclave had a humidity of 100% which is more severe then the humidity (of around 60%) typically found in a Fischer-Tropsch reactor.

### Comparative Example

A first sample (prepared as described above) was subjected to hydrothermal treatment by contact with water in an autoclave at 220° C. for 1 week. The surface area of this catalyst was determined before and after said treatment. Following the treatment the surface area decreased by 17%. Without wishing to be bound by theory this is considered to be as a result of the hydrophilic —OH groups on the carrier surface attracting water which causes agglomeration and sintering of the titania particles, conversion of the anatase titania to rutile titania and oxidation of the active metal; hence a decrease in surface area.

### Example According to the Invention

Two further identical samples (prepared as described above) were treated in accordance with the present invention as detailed below.

During normal calcination, the number of Ti—OH groups is reduced. Thus following calcination, the catalyst samples in this example were treated with water at 90° C. to reverse (in part) this reduction in Ti—OH groups at the surface of the catalyst.

Then the catalyst samples were refluxed with hexamethyldisilazane (HMDS) solution in toluene for 30 mins at a temperature of 144° C.

This results in Si(CH₃)₃ groups replacing the protons on the —OH groups to form the relatively hydrophobic —O—Si(CH₃)₃ groups on the catalyst surface. Ammonia is produced as a by-product. The use of HMDS is particularly beneficial compared with, for example, Si(OCH₃)₄, because inter alia the reaction may be faster, more —OH sites may gain the hydrophobic —O—Si(CH₃)₃ groups and the reaction may be done at a higher temperature.

The process of hydrothermal treatment was then repeated; the pre-treated catalyst samples were subjected to water, and in a separate experiment, water vapour, at 220° C. in an autoclave for a period of 1 week.

In contrast to the untreated catalyst the decrease in surface area was found to be 7% for the pre-treated catalyst subjected to water treatment and 11% for the pre-treated catalyst treated with water vapour.

Thus the addition of the relatively hydrophobic groups by HMDS significantly reduces the loss of surface area for the catalyst pre-treated in accordance with the present invention.

Improvements and modifications may be made without departing from the scope of the invention.

1. A process for modifying a Fischer-Tropsch catalyst or catalyst precursor, the process comprising the steps of:
(a) contacting a Fischer-Tropsch catalyst or catalyst precursor comprising a titania carrier, a catalytically active metal or precursor therefor, and optionally a promoter or precursor therefor, with a compound having the general formula: RₐX₍₄₋ₐ₎Y
wherein a is in the range of 1-3, preferably a=3, and
wherein the or each R is independently an alkyl or aryl group comprising 1 to 8 carbon atoms,
Y is silicon or titanium, and
the or each X is independently chosen from the group consisting of hydrogen, an alkoxy group comprising 1 to 8 carbon atoms, and ZY′BB′B^{2′};
Z being oxygen, —NH, or —NR, wherein R is an alkyl or aryl group comprising 1 to 8 carbon atoms,
Y′ being silicon or titanium, and
B, B′ and B^{2′}, independently being hydrogen or an alkyl, aryl, or alkoxy group comprising 1 to 8 carbon atoms;
(b) keeping the temperature of the resulting modified catalyst or catalyst precursor below 400° C., preferably below 350° C., up to the moment the resulting catalyst or catalyst precursor is subjected to an activation step or used in a Fischer-Tropsch reaction.

2. A process as claimed in claim 1, wherein —Y—RₐX₍₃₋ₐ₎ groups bond to oxygen on the surface of the titania to form O—Y—RₐX₍₃₋ₐ₎.

3. A process as claimed in claim 1, wherein Y is silicon and/or wherein the or each R group in RₐX₍₄₋ₐ₎Y is an alkyl group, preferably a —CH₃ group.

4. A process as claimed in claim 1, wherein a=3 and each R group is a —CH₃ group.

5. A process as claimed in claim 1, wherein a=3 and X is hydrogen or an alkoxy group comprising 1 to 8 carbon atoms.

6. A process as claimed in claim 1, wherein a=3 and X is ZYBB′B^{2′}.

7. A process as claimed in claim 6, wherein Z is —NH.

8. A process as claimed in claim 7, wherein B, B′ and B^{2′} are each an alkyl or aryl group comprising 1 to 8 carbon atoms.

9. A process as claimed in claim 8, wherein the compound is hexamethyldisilazane.

10. A process as claimed in claim 1, wherein before contact with said compound, the catalyst is contacted with H₂O, at a temperature of between 70-110° C.

11. A process for the production of liquid hydrocarbons from synthesis gas, the process comprising:
converting synthesis gas in a reactor into liquid hydrocarbons, and optionally solid hydrocarbons and optionally liquefied petroleum gas, at elevated temperatures and pressures; using a catalyst modified according to claim 1.
